(19) United States
(12) Patent Application Publication
(10) Pub. No.: US 2004/0102488 A1
(43) Pub. Date: May 27, 2004

(54) Butenolide and pentenolide derivatives as kinase inhibitors

(76) Inventors: Barbara Beck, Munchen (DE); Alexander Domling, Munchen (DE)

Correspondence Address: EDWARDS & ANGELL, LLP, P.O. BOX 55874, BOSTON, MA, 02205, US

(21) Appl. No.: 10/399,133

(22) PCT Filed: Oct. 11, 2001
(86) PCT No.: PCT/EP01/11791

(30) Foreign Application Priority Data
Oct. 11, 2000 (DE) 100504906
Oct. 11, 2000 (DE) 100504922

(51) Int. Cl. A61K 31/4439; A61K 31/4415; A61K 31/366; C 07D 4 3/02; C 07D 4 1/02; C07D 213/70; C07D 309/30

(52) U.S. Cl. 514/350; 514/460; 546/290; 546/268.1; 514/340; 549/291

(57) ABSTRACT

The present invention relates to new compounds of the general formula (I),

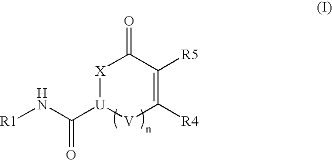

to methods for their preparation, and to pro-drugs, pharmacologically acceptable salts and medicamental compositions comprising them as active ingredient, and to their use as kinase inhibitors.

The present invention describes new butenolide and pentenolide derivatives, processes for their preparation and their use as pharmaceuticals, especially as kinase inhibitors.

The compounds according to the invention are kinase inhibitors and are accordingly of great interest in a very great variety of inflammatory diseases and also in diseases caused by autoimmune reactions, such as, for example, rheumatoid arthritis, asthma, MDR (multiple drug resistance), COPD (chronic obstructive pulmonary disease) and ARDS (acute respiratory distress syndrome), and also in cancer, stroke, Alzheimer's, osteoarthritis, lung disease, septic shock, angiogenesis and dermatitis, and also for in vivo stimulation of nerve growth, in vivo inhibition of scar tissue formation and/or in vivo reduction of secondary damage. Their action allows the active ingredients and pharmaceutical compositions according to the invention to be used as chemotherapeutic agents in human and veterinary medicine.

A further aspect of the present invention is the provision of a new process, for the preparation of those new butenolide and pentenolide derivatives, which as far as possible proceeds in the context of a multicomponent reaction and which is widely applicable. Multicomponent reactions are used especially in the pharmaceutical industry for the production of compound libraries for the finding of lead structures (A. Dömling, I. Ugi, Angew. Chem. 2000, 112, 3300-3344).

The present invention comprises compounds of the general formula (I):

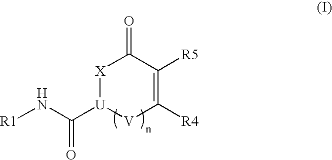

wherein

X is an oxygen atom or a group of formula NR6;

n is 0 or 1;

U is CH or COH;

V is a group of formula CR2R3, or U-V together are a group of formula

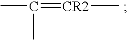

R1 is an alkyl, heteroalkyl, aryl, heteroaryl, cycloalkyl, heterocycloalkyl, aralkyl or heteroaralkyl radical;

R2 is a hydrogen atom, an alkyl, heteroalkyl, aryl, heteroaryl, cycloalkyl, heterocycloalkyl, aralkyl or heteroaralkyl radical;

R3 is a hydrogen atom, an alkyl, heteroalkyl, aryl, heteroaryl, cycloalkyl, heterocycloalkyl, aralkyl or heteroaralkyl radical;

R4 is a hydrogen atom, an alkyl, heteroalkyl, aryl, heteroaryl, cycloalkyl, heterocycloalkyl, aralkyl or heteroaralkyl radical;

R5 is a hydrogen atom, a halogen atom, a hydroxy group, an alkyl, heteroalkyl, aryl, heteroaryl, cycloalkyl, heterocycloalkyl, aralkyl or heteroaralkyl radical;

R6 is a hydrogen atom, an alkyl, heteroalkyl, aryl, heteroaryl, cycloalkyl, heterocycloalkyl, aralkyl or heteroaralkyl radical;

or R2 and R3, or R2 and R4, together are part of a cycloalkyl, heterocycloalkyl, aralkyl or heteroaralkyl ring system;

or a pharmacologically acceptable salt, solvate, hydrate or a pharmacologically acceptable formulation thereof.

In the present invention, terms or parts of terms in the description, claims and abstract are defined as follows:

The expression “alkyl” refers to a saturated or at least partly unsaturated (that is to say, for example, alkenyl or alkynyl), straight-chain or branched hydrocarbon group containing from 1 or 2 to 20 carbon atoms, preferably from 1 or 2 to 12 carbon atoms, especially from 1 or 2 to 6 carbon atoms, for example, the methyl, ethyl, isopropyl, isobutyl, tert-butyl, n-hexyl, 2,2-dimethylbutyl, n-octyl, allyl, isoprenyl or hex-2-enyl group.

The expression “heteroalkyl” refers to an alkyl group in accordance with the above definitions in which one or more carbon atoms (for example, of the saturated or unsaturated chain) has been replaced by at least one oxygen, nitrogen, phosphorus or sulfur atom (preferably oxygen or nitrogen), for example an alkyloxy group such as, for example, methoxy or ethoxy, or a methoxymethyl, nitrile, methylcarboxyalkyl ester, carboxyalkyl ester or 2,3-dioxyethyl group. The expression “heteroalkyl” refers furthermore to a carboxylic acid or a group derived from a carboxylic acid, such as, for example, acyl, acyloxy, carboxyalkyl, carboxyalkyl ester, for example methylcarboxyalkyl ester, carboxyalkylamide, alkoxycarbonyl or alkoxycarbonyloxy.

The expression “cycloalkyl” or “cyclo” refers to a saturated or at least partly unsaturated cyclic group which has one or more rings together containing from 3 to 14 carbon atoms, preferably 3, 4, 5 or 6 to 10 carbon atoms, for example the cyclopropyl, cyclohexyl, Tetralin or cyclohex-2-enyl group.

The expression “heterocycloalkyl” or “heterocyclo” refers to a cycloalkyl group in accordance with the above definitions in which one or more carbon atoms has been replaced by an oxygen, nitrogen, phosphorus or sulfur atom and can denote, for example, the piperidine, morpholine, N-methylpiperazine or N-phenylpiperazine group.

The expression “aryl” or “ar” refers to an aromatic group having one or more rings together containing from 5 to 14 carbon atoms, preferably 5 or 6 to 10 carbon atoms, for example a phenyl, naphthyl, 2-, 3- or 4-methoxyphenyl, 2-, 3- or 4-ethoxyphenyl, 4-carboxyphenylalkyl or 4-hydroxyphenyl group.

The expression “heteroaryl” refers to an aryl group in accordance with the above definitions in which one or more carbon atoms has been replaced by an oxygen, nitrogen, phosphorus or sulfur atom, for example the 4-pyridyl, 2-imidazolyl, 3-pyrazolyl and isoquinolyl group.

The expressions “aralkyl” and “heteroaralkyl” refer to groups including, in accordance with the above definitions, both aryl and heteroaryl and also alkyl and/or heteroalkyl and/or cycloalkyl and/or heterocycloalkyl ring systems, for example the tetrahydroisoquinolyl, benzyl, 2- or 3-ethylindolyl or 4-ethylpyridino group.

The expressions “alkyl”, “heteroalkyl”, “cycloalkyl”, “heterocycloalkyl”, “aryl”, “heteroaryl”, “aralkyl” and “heteroaralkyl” in accordance with the above definitions refer to groups in which one or more hydrogen atoms of such groups have been replaced by fluorine, chlorine, bromine or iodine atoms or by OH, SH, NH₂ or NO₂ groups. Those expressions furthermore refer to groups which are substituted by unsubstituted alkyl, heteroalkyl, cycloalkyl, heterocycloalkyl, aryl, heteroaryl, aralkyl or heteroaralkyl groups.

Compounds of formula (I) may, by virtue of their substitution, contain one or more chiral centres. The present invention accordingly includes all pure enantiomers and all pure diastereomers, and also mixtures thereof in any mixing ratio, and also all tautomers of the compounds described.

Compounds of the general formula (I) wherein X is an oxygen atom are preferred.

Compounds of the general formula (I) wherein X is a group of formula NR6 are also preferred.

Compounds of the general formula (I) wherein U is CH are furthermore preferred.

Compounds of the general formula (I) wherein U is COH are furthermore preferred.

Compounds of the general formula (I) wherein n is 0 are furthermore preferred.

Compounds of the general formula (I) wherein n is 1 are also preferred.

Compounds of the general formula (I) wherein U-V together are a group of formula

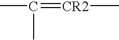

are again preferred.

Compounds of the general formula (I) wherein R5 is not a nitrile group and is not a group of formula R′SO₂—, wherein R′ is an alkyl, heteroalkyl, aryl, heteroaryl, cycloalkyl, heterocycloalkyl, aralkyl or heteroaralkyl radical, are furthermore preferred.

For example, the radicals may have the following meanings:

R2: hydrogen, aryl such as phenyl, heteroaryl

R3: hydrogen, aryl such as phenyl, heteroaryl

R4: aryl such as phenyl, heteroaryl, methyl

R5: hydrogen, aryl such as phenyl, butenyl, fluoro-, chloro- or methoxy-phenyl

Examples of pharmacologically acceptable salts of compounds of formula (I) are salts of physiologically acceptable mineral acids, such as hydrochloric acid, sulfuric acid and phosphoric acid, or salts of organic acids, such as methanesulfonic acid, p-toluenesulfonic acid, lactic acid, acetic acid, trifluoroacetic acid, citric acid, succinic acid, fumaric acid, maleic acid and salicylic acid. Compounds of formula (I) may be solvated, especially hydrated. Hydration may occur, for example, during the preparation process or as a consequence of the hygroscopic nature of the initially anhydrous compounds of formula (I).

The pharmaceutical compositions according to the present invention comprise at least one compound of formula (I) as active ingredient and, optionally, carrier substances and/or adjuvants.

The pro-drugs, to which the present invention also relates, comprise a compound of formula (I) and at least one pharmacologically acceptable protecting group, which is removed under physiological conditions, for example, an alkoxy, aralkyloxy, acyl or acyloxy group, such as, for example, an ethoxy, benzyloxy, acetyl or acetoxy group.

A compound of formula (I) or a pro-drug thereof can be used for inhibiting kinases or for treating and/or preventing diseases mediated by kinase activity. In particular, the compounds according to the invention can be used in a very great variety of inflammatory diseases and also in diseases caused by autoimmune reactions, such as, for example, rheumatoid arthritis, asthma, MDR (multiple drug resistance), COPD (chronic obstructive pulmonary disease) and ARDS (acute respiratory distress syndrome), and also in cancer, stroke, Alzheimer's, osteoarthritis, lung disease, septic shock, angiogenesis and dermatitis, and also for in vivo stimulation of nerve growth, in vivo inhibition of scar tissue formation and/or in vivo reduction of secondary damage. Their action allows the active ingredients and pharmaceutical compositions according to the invention to be used as chemotherapeutic agents in human and veterinary medicine.

The therapeutic use of the compounds of formula (I), their pharmacologically acceptable salts and solvates and hydrates, and formulations and pharmaceutical compositions are also within the scope of the present invention.

The present invention relates also to the use of those active ingredients in the preparation of pharmaceuticals for preventing and/or treating diseases mediated by kinase activity. In general, compounds of formula (I) are administered either on their own or in combination with any other therapeutic composition, using known and acceptable means. Such therapeutically useful compositions can be administered by one of the following routes: orally, for example in the form of dragees, coated tablets, pills, semi-solid substances, soft or hard capsules, solutions, emulsions or suspensions; parenterally, for example in the form of an injectable solution; rectally, in the form of suppositories; by inhalation, for example in the form of a powder formulation or spray; transdermally or intranasally. For the preparation of such tablets, pills, semi-solid substances, coated tablets, dragees and hard gelatin capsules, the therapeutically useful product can be mixed with pharmacologically inert, inorganic or organic pharmaceutical carrier substances, for example with lactose, sucrose, glucose, gelatin, malt, silica gel, starch or derivatives thereof, talc, stearic acid or salts thereof, dry skimmed milk and the like. For the preparation of soft capsules, pharmaceutical carrier substances, such as, for example, vegetable oils, petroleum, animal or synthetic oils, wax, fat or polyols may be used. For the preparation of liquid solutions and syrups, pharmaceutical carrier substances, such as, for example, water, alcohols, aqueous salt solution, aqueous dextrose, polyols, glycerol, vegetable oils, petroleum, animal or synthetic oils may be used. For suppositories, pharmaceutical carrier substances, such as, for example, vegetable oils, petroleum, animal or synthetic oils, wax, fat and polyols may be used. For aerosol formulations, compressed gases that are suitable for the purpose, such as, for example, oxygen, nitrogen and carbon dioxide may be used. The pharmaceutically useful compositions may also comprise additives for preservation, stabilisation, emulsifiers, sweeteners, aromas, salts for modifying osmotic pressure, buffers, coating additives and antioxidants.

Combinations with other therapeutic compositions may comprise other active ingredients which are conventionally used for the prevention and/or treatment of diseases mediated by kinase activity.

Compounds of formula (I) wherein X is an oxygen atom can be prepared in accordance with the invention by reacting compounds of the general formulae (II), (III) and (IV)

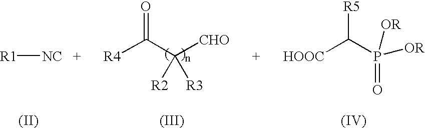

the radicals being as defined hereinbefore.

Compounds of formula (I) wherein X is a group of formula NR6 can be prepared in accordance with the invention by reacting compounds of the general formulae (II), (III) (IV) and (V).

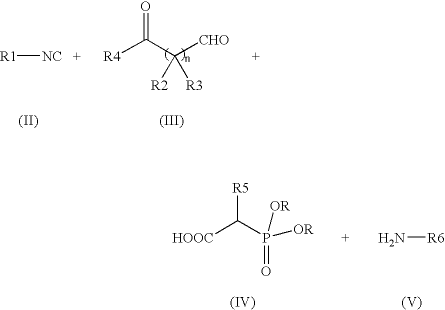

The process described proceeds in two steps, the first step corresponding to a Passerini or Ugi reaction and the second step corresponding to an intramolecular Horner-Wittig-Emmons reaction. The two steps of the process can be carried out either separately or in a one-pot reaction.

If the above-described reactions are carried out with the exclusion of O₂ (for example under an N₂ or argon atmosphere), there are obtained compounds of formula (VI):

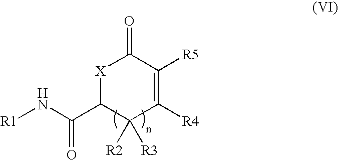

If the above-described reactions are carried out in the presence of O₂, there are obtained, when neither R2 nor R3 nor R6 is a hydrogen atom, compounds of the general formula (VII):

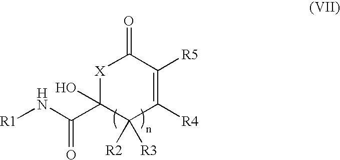

In the case where R6=H, the corresponding dihydropyridines are formed, the present invention also relating thereto.

If R2 is a hydrogen atom and n is 1, there are obtained, in the above-described reactions, compounds of the general formula (VIII):

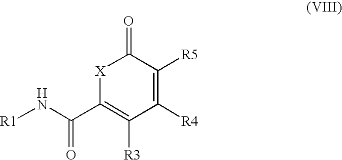

Preference is given to carrying out the reactions in aprotic solvents, such as, for example, dimethoxyethane, acetonitrile, dichloromethane, chloroform, toluene, benzene, diethyl ether or tetrahydrofuran, preferably in ethers.

Preference is also given to carrying out the reactions in a temperature range from −80 to 120° C., more preferably from −10 to 70° C., and even more preferably from 0° C. to 25° C.

Special preference is given to carrying out the second step in the presence of alkali metal salts, such as, for example, LiCl, LiBr, Cs₂CO₃ or K₂CO₃, with special preference being given to lithium salts, such as, for example, LiCl.

Preference is furthermore given to alternatively or additionally carrying out the second step of the reaction in the presence of a base such as, for example, sodium hydride, potassium tert-butanolate, n-butyl lithium, lithium diisopropylamide, potassium carbonate/18-crown-6, potassium hexamethyldisilazide (KHDMS)/18-crown-6, triethylamine, diisopropylethylamine (DIPEA), diazabicycloundecene (DBU) or 4-dimethylaminopyridine (DMAP) or diazabicyclononene (DBN); preferably a tertiary, basic amine.

### EXAMPLES

Proton and carbon NMR spectra were measured in the solvent indicated in each case, using a Mercury 400 spectrometer. The chemical shifts (δ) are given in ppm in relation to TMS as internal standard. The electrospray ionisation (ESI) mass spectra were recorded using an MSD (Hewlett-Packard's HPLC 1100-assisted electrospray MS instrument). In each case the observed m/z ratios are given as [M+H]⁺ or [M+Na]⁺ signals. The products were purified by means of preparative chromatography using silica gel, and ethyl acetate as eluant. The purity was determined using a Hewlett-Packard LC 1100 system (YMC column, 2 mm x 50 mm, 2 pm ODSA, 220 and 254 nm; 0.6 ml/min., 6 min. gradient from 90% H₂O to 10% H₂O (0.5% CH₃COOH) versus CH₃CN.). In each case the retention time t_{R} is given in minutes, at the wavelength 254 nm.

### General Work Procedures

### General Procedure for the Passerini Reaction

2 mmol of the aldehyde (III) are suspended in 4 ml of diethyl ether, and 2 mmol of the phosphonoacetic acid (IV) are added. The reaction mixture is stirred at room temperature for five minutes. 2 mmol of the isocyanide (II) are then added and the reaction mixture is stirred at room temperature overnight. The solvent is then removed using a rotary evaporator.

### General Procedure for the Ugi Reaction

2 mmol of the aldehyde (III) are dissolved in 4 ml of dichloromethane, and 2 mmol of the amine (V) are added. 500 mg of 4 Å molecular sieve are added to the reaction mixture and stirring is carried out at RT overnight. The molecular sieve is filtered off and the solution is concentrated. The residue is taken up in 2 ml of methanol, and 2 mmol of the phosphonoacetic acid (IV) and 2 mmol of the isocyanide (II) are added. The reaction mixture is stirred at room temperature overnight. The solvent is drawn off, and the Ugi product is purified by means of column chromatography.

### General Procedure for the Horner-Wittig-Emmons Ring Closure

The reaction is carried out, where appropriate, under a nitrogen atmosphere. 1.5 mmol of the Passerini or Ugi product prepared above are dissolved in 60 ml of THF; 4.5 mmol of dry lithium chloride are added and stirring is carried out at 0° C. for 5 minutes, whereupon the salt dissolves completely. 15 mmol of triethylamine are added to the resulting solution dropwise and stirring is carried out at 0° C. for a further 15 minutes. The reaction mixture is then allowed to warm up to room temperature and is stirred for a further 4 hours. The reaction mixture is filtered through a silica gel layer, washing with 180 ml of ethyl acetate. The solvent is drawn off and the residue is recrystallised from ethyl acetate.

### General Procedure for the One-Pot Reaction of the Passerini Variant

The reaction is, where appropriate, carried out under a nitrogen atmosphere. 2 mmol of the aldehyde (III) are suspended in 4 ml of THF. 2 of the phosphonoacetic acid (IV) are added to the resulting solution and stirring is carried out at RT for 5 minutes, whereupon a clear solution is obtained. 2 mmol of the isocyanide (II) are added to the resulting solution and the reaction mixture is stirred at room temperature overnight. 60 ml of THF and 4.5 mmol of lithium chloride are added to the reaction mixture and stirring is carried out at 0° C. for 5 minutes. After the salt has dissolved completely, 15 mmol of triethylamine are slowly added dropwise and stirring is carried out at 0° C. for a further 15 minutes. The solution is allowed to warm up to RT and is stirred for a further 4 hours. The reaction mixture is filtered through a silica gel layer, washing with 180 ml of ethyl acetate. The solvent is drawn off and the residue is recrystallised from ethyl acetate.

### General Procedure for the One-Pot Reaction of the Ugi Variant

The reaction is carried out, where appropriate, under a nitrogen atmosphere. 2 mmol of the aldehyde (III) are dissolved in 4 ml of dichloromethane, and 2 mmol of the amine (V) are added. 500 mg of 4 Å molecular sieve are added to the resulting reaction solution and stirring is carried out at RT overnight. The molecular sieve is filtered off and the solution is concentrated. The residue is taken up in 2 ml of methanol, and 2 mmol of the phosphonoacetic acid (IV) and 2 mmol of the isocyanide (II) are added. The reaction mixture is stirred at room temperature overnight. The solvent is drawn off and the residue is taken up in 60 ml of THF; 4.5 mmol of lithium chloride are added and stirring is carried out at 0° C. for 5 minutes. After the salt has dissolved completely, 15 mmol of triethylamine are slowly added dropwise and stirring is carried out at 0° C. for a further 15 minutes. The solution is allowed to warm up to RT and stirring is carried out for a further 4 hours. The reaction mixture is filtered through a silica gel layer, washing with 180 ml of ethyl acetate. The solvent is drawn off and the residue is recrystallised from ethyl acetate.

### Example 1

### Preparation of 5-oxo-3-phenyl-2,5-dihydro-furan-2-carboxylic acid tert-butylamide

| | | |
|---|---|---|
| Empirical formula: Molecular weight: Yield: | C₁₅H₁₇NO₃259.31 g/mol 226 mg (87% of theory) | 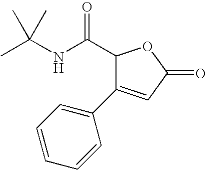 |

HPLC-MS (ESI-TOF): t_{R, 254 nm}=3.359 min; m/z=260 [M+H]⁺; 282 [M+Na]⁺.

¹H NMR (CDCl₃, 400 MHz): δ=7.74 (m, 2H, a1, a2, aromatic), 7.46 (m, 3H, b1, b2, c, aromatic), 6.31 (s, 1H, (C)CH(CO)), 6.19 (broad s, 1H, NH), 5.79 (s, 1H, (C═O)CH(C—O)), 1.29 (s, 9H, 3CH3).

¹³C NMR (CDCl₃, 100 MHz): δ=171.71 (CO), 165.68 (CO), 163.76 (CO), 131.68 (2CH, aromatic), 129.37 (C, aromatic), 128.61 (2CH, aromatic), 128.52 (CH, aromatic), 113.05 (C═CH(CO)), 81.40 ((CO) CH—O), 51.92 ((CH)(Phe)C═CH), 28.38 (3CH3, tBu).

### Example 2

### Preparation of 3-(4-hydroxy-phenyl)-5-oxo-2,5-dihydro-furan-2-carboxylic acid (7-isopropyl-1,4a-dimethyl-1,2,3,4,4a,9,10,10a-octahydro-phenanthren-1-ylmethyl)-amide

| | | |
|---|---|---|
| Empirical formula: Molecular weight: | C₃₁H₃₇NO₄ 487.64 g/mol | 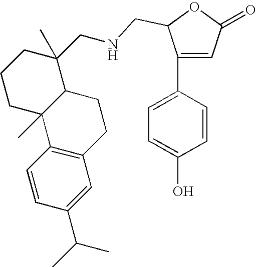 |

HPLC-MS (ESI-TOF): t_{R}=4.204 min; m/z=642 [M+H]⁺;

### Example 3

### Preparation of 3-biphenyl-4-yl-5-oxo-2,5-dihydro-furan-2-carboxylic acid benzhydryl-amide

| | | |
|---|---|---|
| Empirical formula: Molecular weight: | C₃₀H₂₃NO₃ 445.52 g/mol | 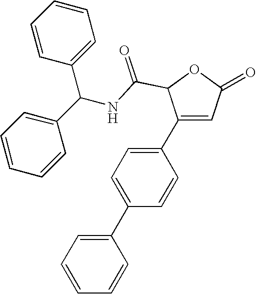 |

HPLC-MS (ESI-TOF): t_{R}=4.070 min; m/z=468 [M+Na]⁺.

### Example 4

### Preparation of 2-hydroxy-5-oxo-3,4-diphenyl-2,5-dihydro-furan-2-carboxylic acid cyclohexylamide

| | | |
|---|---|---|
| Empirical formula: Molecular weight: | C₂₃H₂₃NO₄ 377.44 g/mol | 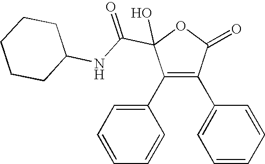 |

HPLC-MS (ESI-TOF): t_{R}=3.764 min; m/z=378 [M+H]⁺; 400 [M+Na]⁺.

### Example 5

### Preparation of [(5-oxo-3-phenyl-2,5-dihydro-furan-2-carbonyl)-amino]-acetic acid tert-butyl ester

| | | |
|---|---|---|
| Empirical formula: Molecular weight: | C₁₇H₁₉NO₅ 317.34 g/mol | 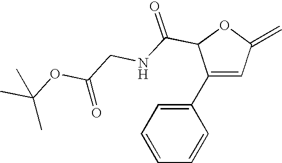 |

HPLC-MS (ESI-TOF): t_{R}=3.449 min; m/z=340 [M+Na]⁺.

### Example 6

### Preparation of [(2-hydroxy-5-oxo-3,4-diphenyl-2,5-dihydro-furan-2-carbonyl)-amino]-acetic acid tert-butyl ester

| | | |
|---|---|---|
| Empirical formula: Molecular weight: | C₂₃H₂₃NO₆409.44 g/mol | 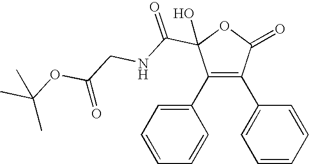 |

HPLC-MS (ESI-TOF): t_{R}=3.693 min; m/z=432 [M+Na]⁺.

### Example 7

### Preparation of [(2-hydroxy-5-oxo-3,4-diphenyl-2,5-dihydro-furan-2-carbonyl)-amino]-phenyl-acetic acid tert-butyl ester

| | | |
|---|---|---|
| Empirical formula: Molecular weight: | C₂₉H₂₇NO₆485.54 g/mol | 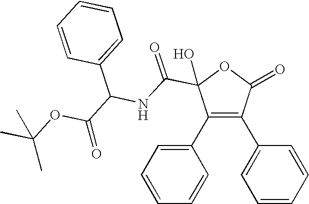 |

HPLC-MS (ESI-TOF): t_{R}=3.970 min; m/z=486 [M+H]⁺; 508 [M+Na]⁺.

### Example 8

### Preparation of [(5-oxo-3,4-diphenyl-2,5-dihydro-furan-2-carbonyl)-amino]-phenyl-acetic acid tert-butyl ester

| | | |
|---|---|---|
| Empirical formula: Molecular weight: | C₂₉H₂₇NO₅469.54 g/mol | 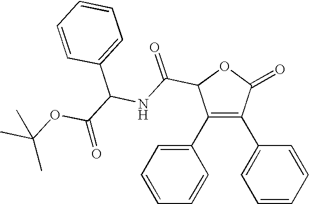 |

HPLC-MS (ESI-TOF): t_{R}=4.003 min; m/z=492 [M+Na]⁺.

### Example 9

### Preparation of 5-oxo-3,4-diphenyl-2,5-dihydro-furan-2-carboxylic acid tert-butylamide

| | | |
|---|---|---|
| Empirical formula: Molecular weight: Yield: | C₂₁H₂₁NO₃335.41 g/mol 36% of theory | 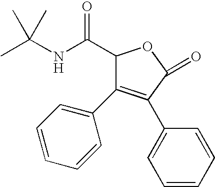 |

HPLC-MS (ESI-TOF): t_{R}=3.659 min; m/z=336 [M+H]⁺; 358 [M+Na]⁺.

¹H NMR (CDCl₃, 400 MHz): δ=7.29 (m, 10H, CH aromatic), 6.08 (broad s, 1H, NH), 5.63 (s, 1H, (CO)CH(O)), 1.19 (s, 9H, 3×CH3).

¹³C NMR (CDCl₃, 100 MHz): δ=171.76 (CO), 163.59 (CO), 158.13 (CO), 130.37 (CH, aromatic), 130.27 (C, aromatic), 129.26 (CH, aromatic), 128.89 (CH, aromatic), 128.82 (CH, aromatic), 128.46 (CH, aromatic), 128.40 (CH, aromatic), 125.7 ((Phe)C(CH) or ((Phe)C(CO) butenolide ring), 81.02 ((CO)CH(O)), 51,87 (C(Me)3, tBu), 28.40 (3CH3, tBu).

### Example 10

### Preparation of 3-naphth-2-yl-5-oxo-2,5-dihydro-furan-2-carboxylic acid allylamide

| | | |
|---|---|---|
| Empirical formula: Molecular weight: Yield: | C₁₈H₁₅NO₃293.33 g/mol 329 mg (74% of theory) | 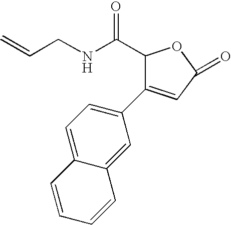 |

HPLC-MS (ESI-TOF): t_{R}=3.356 min; m/z=294 [M+H]⁺; 316 [M+Na]⁺.

¹H NMR (CDCl₃, 400 MHz): δ=8.39 (s, 1H, CH aromatic), 7.95 (d, 1H, CH aromatic), 7.88 (d, 1H, CH aromatic), 7.84 (d, 1H, CH aromatic) 7.73 (dd, 1H, CH aromatic), 7.55 (qd, 2H, 2×CH aromatic), 6.68 (broad s, 1H, NH), 6.44 (s, 1H, (CO)CH(O)), 6.05 (s, 1H, (C)═CH(CO)), 5.75 (m, 1H, (CH2═CH(CH2)), 5.15 (t, 2H, CH2═CH), 3.89 (dm, 1H, 1H of (CH)CH2(NH)), 3.75 (dm, 1H, 1H of (CH)CH2(NH)).

¹³C NMR (CDCl₃, 100 MHz): δ=171.82 (CO), 165.22 (CO), 165.01 (CO), 134.68 (C, aromatic), 132.86 (C, aromatic), 132.55 (C, aromatic), 130.13 (CH, aromatic), 129.30 (CH, aromatic), 128.47 (CH, aromatic), 128.23 (CH, aromatic), 127.66 (CH, aromatic), 126.95 (CH, aromatic), 126.26 (CH, aromatic), 124.49 (CH═CH2), 117.16 (CH2═CH), 112.95 (C═CH(CO)), 81.16 ((CO) CH—O), 41.80 (CH2).

### Example 11

### Preparation of 3-biphenyl-4-yl-5-oxo-2,5-dihydro-furan-2-carboxylic acid butylamide

| | | |
|---|---|---|
| Empirical formula: Molecular weight: Yield: | C₂₁H₂₁NO₃ 335.41 g/mol 427 mg (85% of theory) | 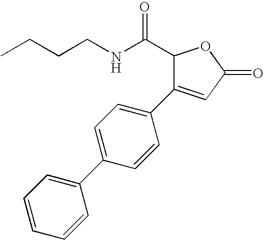 |

HPLC-MS (ESI-TOF): t_{R}=3.352 min; m/z=294 [M+H]⁺; 316 [M+Na]⁺.

¹H NMR (CDCl₃, 400 MHz): δ=7.86 (d, 2H, a1, a2, aromatic), 7.68 (d, 2H, b1, b2, aromatic), 7.61 (d, 2H, c1, c2, aromatic), 7.46 (t, 2H, d1, d2, aromatic), 7.39 (t, 1H, e, aromatic), 6.64 (broad s, 1H, NH), 6.34 (s, 1H, (C)CH(CO)), 5.93 (s, 1H, (C═O)CH(C—O)), 3.29 (m, 1H, (CH2)CH2(NH)), 3.15 (m, 1H, (CH2)CH2(NH)), 1.45 (td, 2H, CH2CH2CH2), 1.28 (m, 2H, CH3CH2), 0.87 (t, 3H, CH3).

¹³C NMR (CDCl₃, 100 MHz): δ=171.93 (CO), 165.08 (CO), 164.89 (C, aromatic), 144.54 (C, aromatic), 139.65 (C, aromatic), 129.20 (CH, aromatic), 128.88 (CH, aromatic), 128.10 (CH, aromatic), 127.93 (CH, aromatic), 112.49 (C═CH(CO)), 81.15 ((CO) CH—O), 39.28 ((CH2)CH2(NH)), 31.14 ((CH2)CH2(CH2)), 19.85 ((CH3)CH2), 13.57 (CH3).

### Example 12

### Preparation of 3-{[3-(4-hydroxy-phenyl)-5-oxo-2,5-dihydro-furan-2-carbonyl]-amino}-propionic acid tert-butyl ester

| | | |
|---|---|---|
| Empirical formula: Molecular weight: Yield: Appearance: | C₁₈H₂₁NO₆347.37 g/mol 565 mg (81% of theory) yellow foam | 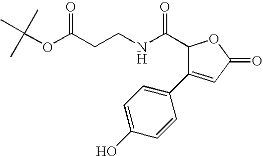 |

HPLC-MS (ESI-TOF): t_{R}=3.110 min; m/z=370 [M+Na]⁺.

¹H NMR (DMSO, 400 MHz): δ=8.78 (s, 1H, OH), 7.58 (d, 2H, 2×CH aromatic), 6.81 (d, 2H, 2×CH aromatic), 6.55 (s, 1H, (CO)(CO)CH(C—O)), 5.93 (s, 1H, (C)═CH(CO)), 3.29 (m, 2H, (CH2)CH2(NH)), 2.48, 2.27 (m, 2H, ((CO)CH2(CH2)), 1.37 (s, 9H, 3×CH3 tBu).

¹³C NMR (DMSO, 100 MHz): δ=172.95 (CO), 170.30 (CO), 165.11 (CO), 163.52 (CO), 160.58 (C(OH), aromatic), 129.52 (2×CH, aromatic), 120.14 (1C, aromatic), 115.60 (2×CH, aromatic), 110.56 (C═CH(CO)), 80.36 ((CO)CH(O)), 35.10 (CH2), 34.28 (CH2), 27.66 (3CH3, tBu).

### Example 13

### Preparation of 4-naphth-2-yl-6-oxo-6H-pyran-2-carboxylic acid tert-butylamide

| | | |
|---|---|---|
| Empirical formula: Molecular weight: | C₂₀H₁₉NO₃321.38 g/mol | 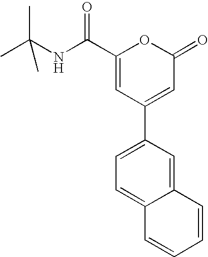 |

HPLC-MS (ESI-TOF): t_{R}=3.747 min; m/z=282 [M−CO₂+H]⁺; 304 [M−CO₂+Na]⁺.

### Example 14

### Preparation of 4-tert-butyl-6-oxo-6H-pyran-2-carboxylic acid tert-butylamide

| | | |
|---|---|---|
| Empirical formula: Molecular weight: | C₁₄H₂₁NO₃251.33 g/mol | 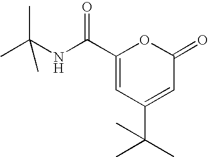 |

HPLC-MS (ESI-TOF): t_{R}=3.424 min; m/z=212 [M−CO₂+H]⁺; 234 [M−CO₂+Na]⁺.

### Example 15

### Preparation of 4-(4-fluoro-phenyl)-6-oxo-6H-pyran-2-carboxylic acid tert-butylamide

| | | |
|---|---|---|
| Empirical formula: Molecular weight: | C₁₆H₁₆FNO₃289.31 g/mol | 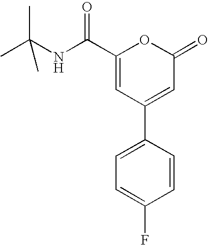 |

HPLC-MS (ESI-TOF): t_{R}=3.471 min; m/z=250 [M−CO₂+H]⁺; 272 [M−CO₂+Na]⁺.

### Example 16

### Preparation of 4-biphenyl-4-yl-6-oxo-6H-pyran-2-carboxylic acid tert-butylamide

| | | |
|---|---|---|
| Empirical formula: Molecular weight: | C₂₂H₂₁NO₃347.42 g/mol | 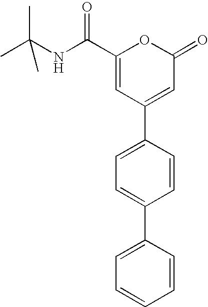 |

HPLC-MS (ESI-TOF): t_{R}=3.863 min; m/z=308 [M−CO₂+H]⁺; 330 [M−CO₂+Na]⁺.

### Example 17

### Preparation of 4-(3-hydroxy-phenyl)-6-oxo-6H-pyran-2-carboxylic acid tert-butylamide

| | | |
|---|---|---|
| Empirical formula: Molecular weight: | C₁₆H₁₇NO₄287.32 g/mol | 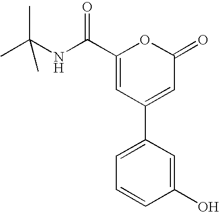 |

HPLC-MS (ESI-TOF): t_{R}=3.275 min; m/z=248 [M−CO₂+H]⁺; 270 [M−CO₂+Na]⁺.

### Example 18

### Preparation of 3-oxo-5,6,7,8-tetrahydro-3H-isochromene-1-carboxylic acid tert-butylamide

| | | |
|---|---|---|
| Empirical formula: Molecular weight: | C₁₄H₁₉NO₃249.31 g/mol | 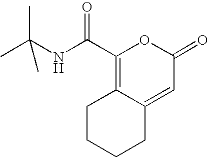 |

HPLC-MS (ESI-TOF): t_{R}=3.191 min; m/z=210 [M−CO₂+H]⁺; 232 [M−CO₂+Na]⁺.

### Example 19

### Preparation of 3-oxo-9,10-dihydro-3H-2-oxa-phenanthrene-1-carboxylic acid tert-butylamide

| | | |
|---|---|---|
| Empirical formula: Molecular weight: | C₁₈H₁₉NO₃297.36 g/mol | 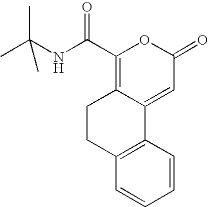 |

HPLC-MS (ESI-TOF): t_{R}=3.582 min; m/z=258 [M−CO₂+H]⁺; 280 [M−CO₂+Na]⁺.

### Example 20

### Preparation of 6-oxo-3,4-diphenyl-6H-pyran-2-carboxylic acid tert-butylamide

| | | |
|---|---|---|
| Empirical formula: Molecular weight: | C₂₂H₂₁NO₃347.42 g/mol | 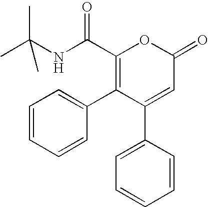 |

HPLC-MS (ESI-TOF): t_{R}=3.687 min; m/z=308 [M−CO₂+H]⁺; 330 [M−CO₂+Na]⁺.

### Example 21

### Preparation of 3,4-bis(4-methoxy-phenyl)-6-oxo-6H-pyran-2-carboxylic acid tert-butylamide

| | | |
|---|---|---|
| Empirical formula: Molecular weight: | C₂₄H₂₅NO₅ 407.47 g/mol | 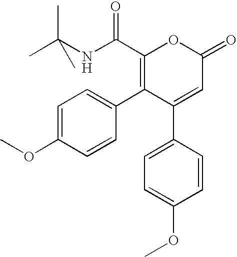 |

HPLC-MS (ESI-TOF): t_{R}=3.627 min; m/z=368 [M−CO₂+H]⁺; 390 [M−CO₂+Na]⁺.

### Example 22

### Preparation of 5-oxo-3-phenyl-2,5-dihydro-furan-2-carboxylic acid {[2-(3H-isoindol-1-yl)-ethylcarbamoyl]-methyl}-amide

| | | |
|---|---|---|
| Empirical formula: Molecular weight: | C₂₃H₂₁N₃O₄403.44 g/mol | 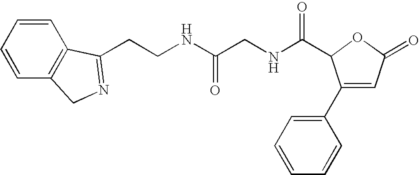 |

HPLC-MS (ESI-TOF): t_{R}=3.249 min; m/z=404 [M+H]⁺; 426 [M+Na]⁺.

### Example 23

### Preparation of 5-oxo-3-phenyl-2,5-dihydro-furan-2-carboxylic acid butylamide

| | | |
|---|---|---|
| Empirical formula: Molecular weight: | C₁₅H₁₇NO₃ 259.31 g/mol | 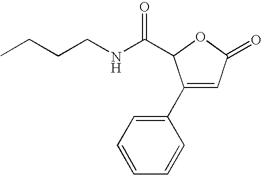 |

HPLC-MS (ESI-TOF): t_{R}=3.320 min; m/z=260 [M+H]⁺; 282 [M+Na]⁺.

### Example 24

### Preparation of 5-oxo-3-phenyl-2,5-dihydro-furan-2-carboxylic acid benzhydryl-amide

| | | |
|---|---|---|
| Empirical formula: Molecular weight: | C₂₄H₁₉NO₃ 369.42 g/mol | 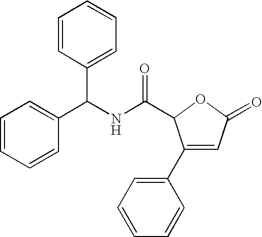 |

HPLC-MS (ESI-TOF): t_{R}=3.625 min; m/z=370 [M+H]⁺; 392 [M+Na]⁺.

### Example 25

### Preparation of 5-oxo-3-phenyl-2,5-dihydro-furan-2-carboxylic acid benzylamide

| | | |
|---|---|---|
| Empirical formula: Molecular weight: | C₁₈H₁₅NO₃ 293.33 g/mol | 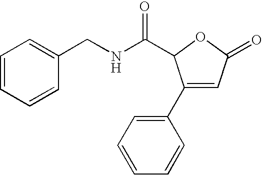 |

HPLC-MS (ESI-TOF): t_{R}=3.311 min; m/z=294 [M+H]⁺; 316 [M+Na]⁺.

### Example 26

### Preparation of 3-biphenyl-4-yl-5-oxo-2,5-dihydro-furan-2-carboxylic acid cyclohexylamide

| | | |
|---|---|---|
| Empirical formula: Molecular weight: | C₂₃H₂₃NO₃ 361.44 g/mol | 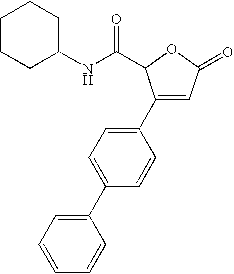 |

HPLC-MS (ESI-TOF): t_{R}=3.778 min; m/z=362 [M+H]⁺; 384 [M+Na]⁺.

### Example 27

### Preparation of 3-biphenyl-4-yl-5-oxo-2,5-dihydro-furan-2-carboxylic acid tert-butylamide

| | | |
|---|---|---|
| Empirical formula: Molecular weight: | C₂₁H₂₁NO₃335.41 g/mol | 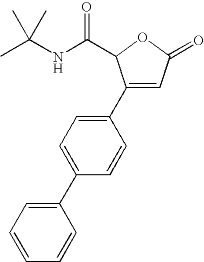 |

HPLC-MS (ESI-TOF): t_{R}=3.688 min; m/z=336 [M+H]⁺; 358 [M+Na]⁺.

### Example 28

### Preparation of 3-biphenyl-4-yl-5-oxo-2,5-dihydro-furan-2-carboxylic acid benzylamide

| | | |
|---|---|---|
| Empirical formula: Molecular weight: | C₂₄H₁₉NO₃ 369.42 g/mol | 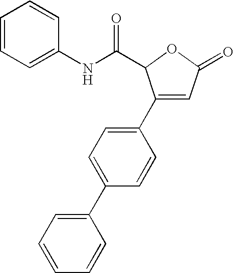 |

HPLC-MS (ESI-TOF): t_{R}=3.712 min; m/z=370 [M+H]⁺; 392 [M+Na]⁺.

### Example 29

### Preparation of 3-(4-nitro-phenyl)-5-oxo-2,5-dihydro-furan-2-carboxylic acid benzhydryl-amide

| | | |
|---|---|---|
| Empirical formula: Molecular weight: | C₂₄H₁₈N₂O₅ 414.42 g/mol | 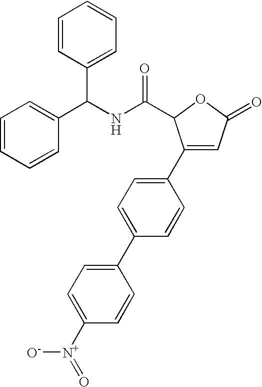 |

HPLC-MS (ESI-TOF): t_{R}=3.866 min; m/z=415 [M+H]⁺;

### Example 30

### Preparation of 4-(3-cyano-phenyl)-6-oxo-6H-pyran-2-carboxylic acid tert-butylamide

| | | |
|---|---|---|
| Empirical formula: Molecular weight: | C₁₇H₁₆N₂O₃ 296.33 g/mol | 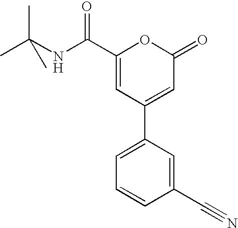 |

HPLC-MS (ESI-TOF): t_{R}=3.481 min; m/z=257 [M−CO₂+H]⁺; 279 [M−CO₂+Na]⁺.

### Example 31

### Preparation of 3-(4-cyano-phenyl)-5-oxo-2,5-dihydro-furan-2-carboxylic acid cyclohexylamide

| | | |
|---|---|---|
| Empirical formula: Molecular weight: | C₁₈H₁₈N₂O₃310.36 g/mol | 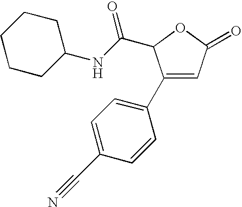 |

### Example 32

### Preparation of 3-naphth-1-yl-5-oxo-2,5-dihydro-furan-2-carboxylic acid butylamide

| | | |
|---|---|---|
| Empirical formula: Molecular weight: | C₁₉H₁₉NO₃309.37 g/mol | 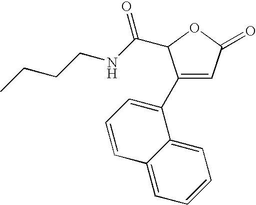 |

HPLC-MS (ESI-TOF): t_{R}=3.517 min; m/z=310 [M−CO₂+H]⁺; 332 [M+Na]⁺.

### Example 33

### Preparation of 3-naphth-1-yl-5-oxo-2,5-dihydro-furan-2-carboxylic acid cyclohexylamide

| | | |
|---|---|---|
| Empirical formula: Molecular weight: | C₂₁H₂₁NO₃335.41 g/mol | 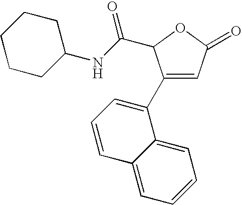 |

HPLC-MS (ESI-TOF): t_{R}=3.638 min; m/z=336 [M+H]⁺; 358 [M+Na]⁺.

### Example 34

### Preparation of 3-naphth-1-yl-5-oxo-2,5-dihydro-furan-2-carboxylic acid tert-butylamide

| | | |
|---|---|---|
| Empirical formula: Molecular weight: | C₁₉H₁₉NO₃309.37 g/mol | 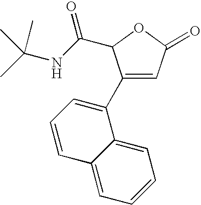 |

HPLC-MS (ESI-TOF): t_{R}=3.524 min; m/z=310 [M+H]⁺; 332 [M+Na]⁺.

### Example 35

### Preparation of 3-naphth-1-yl-5-oxo-2,5-dihydro-furan-2-carboxylic acid benzylamide

| | | |
|---|---|---|
| Empirical formula: Molecular weight: | C₂₂H₁₇NO₃343.39 | 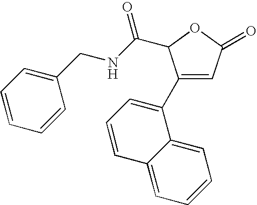 |

HPLC-MS (ESI-TOF): t_{R}=3.545 min; m/z=344 [M+H]⁺; 366 [M+Na]⁺.

### Example 36

### Preparation of 3-(4-chloro-phenyl)-5-oxo-2,5-dihydro-furan-2-carboxylic acid {[2-(1H-indol-3-yl)-ethylcarbamoyl]-methyl}-amide

| | | |
|---|---|---|
| Empirical formula: Molecular weight: | C₂₃H₂₀ClN₃O₄437.89 g/mol | 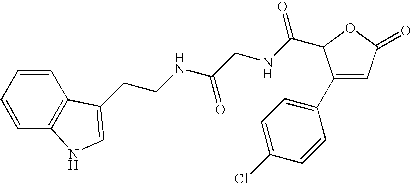 |

HPLC-MS (ESI-TOF): t_{R}=3.358 min; m/z=460 [M+Na]⁺.

### Example 37

### Preparation of 3-(4-chloro-phenyl)-5-oxo-2,5-dihydro-furan-2-carboxylic acid butylamide

| | | |
|---|---|---|
| Empirical formula: Molecular weight: | C₁₅H₁₆ClNO₃293.75 g/mol | 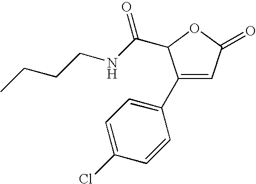 |

HPLC-MS (ESI-TOF): t_{R}=3.453 min; m/z=294 [M+H]⁺; 316 [M+Na]⁺.

### Example 38

### Preparation of 3-(4-chloro-phenyl)-5-oxo-2,5-dihydro-furan-2-carboxylic acid cyclohexylamide

| | | |
|---|---|---|
| Empirical formula: Molecular weight: | C₁₇H₁₈ClNO₃319.79 g/mol | 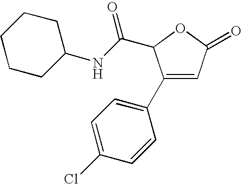 |

HPLC-MS (ESI-TOF): t_{R}=3.581 min; m/z=320 [M+H]⁺; 342 [M+Na]⁺.

### Example 39

### Preparation of 3-(4-chloro-phenyl)-5-oxo-2,5-dihydro-furan-2-carboxylic acid tert-butylamide

| | | |
|---|---|---|
| Empirical formula: Molecular weight: | C₁₅H₁₆ClNO₃293.75 g/mol | 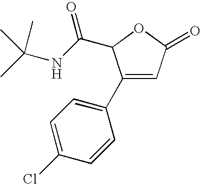 |

HPLC-MS (ESI-TOF): t_{R}=3.452 min; m/z=294 [M+H]⁺; 316 [M+Na]⁺.

### Example 40

### Preparation of 3-(4-chloro-phenyl)-5-oxo-2,5-dihydro-furan-2-carboxylic acid allylamide

| | | |
|---|---|---|
| Empirical formula: Molecular weight: | C₁₄H₁₂ClNO₃277.71 g/mol | 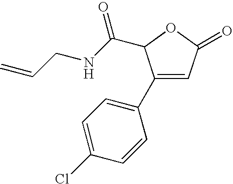 |

HPLC-MS (ESI-TOF): t_{R}=3.252 min; m/z=278 [M+H]⁺; 300 [M+Na]⁺.

### Example 41

### Preparation of 3-(4-chloro-phenyl)-5-oxo-2,5-dihydro-furan-2-carboxylic acid benzhydryl-amide

| | | |
|---|---|---|
| Empirical formula: Molecular weight: | C₂₄H₁₈ClNO₃ 403.87 g/mol | 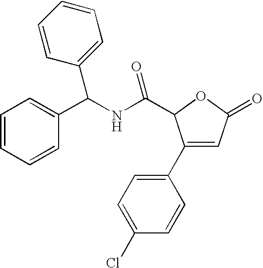 |

HPLC-MS (ESI-TOF): t_{R}=3.759 min; m/z=404 [M+H]⁺; 426 [M+Na]⁺.

### Example 42

### Preparation of 3-(4-chloro-phenyl)-5-oxo-2,5-dihydro-furan-2-carboxylic acid benzylamide

| | | |
|---|---|---|
| Empirical formula: Molecular weight: | C₁8H₁₄ClNO₃ 327.77 g/mol | 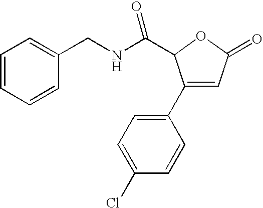 |

HPLC-MS (ESI-TOF): t_{R}=3.487 min; m/z=328 [M+H]⁺; 350 [M+Na]⁺.

### Example 43

### Preparation of 1-(3-cyano-phenyl)-3-methyl-5-oxo-2,5-dihydro-1H-pyrrole-2-carboxylic acid (3-phenoxy-phenyl)-amide

| | | |
|---|---|---|
| Empirical formula: Molecular weight: | C₂₅H₁₉N₃O₃409.45 g/mol | 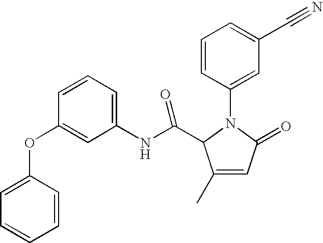 |

HPLC-MS (ESI-TOF): t_{R}=3.805 min; m/z=410 [M+H]⁺; 432 [M+Na]⁺.

### Example 44

### Preparation of 1-allyl-3-naphth-2-yl-5-oxo-2,5-dihydro-1H-pyrrole-2-carboxylic acid (3-benzoyl-phenyl)-amide

| | | |
|---|---|---|
| Empirical formula: Molecular weight: | C₁₃H₂₄N₂O₃472.55 g/mol | 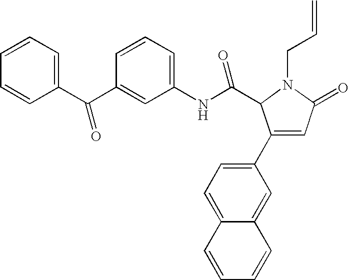 |

HPLC-MS (ESI-TOF): t_{R}=3.890 min; m/z=473 [M+H]⁺; 495 [M+Na]⁺.

### Example 45

### Preparation of 1-cyclopropyl-3-naphth-2-yl-5-oxo-2,5-dihydro-1H-pyrrole-2-carboxylic acid (pyrid-3-ylmethyl)-amide

| | | |
|---|---|---|
| Empirical formula: Molecular weight: | C₂₇H₃₁N₅O₃ 383.45 g/mol | 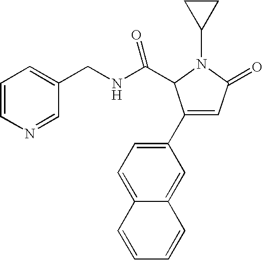 |

HPLC-MS (ESI-TOF): t_{R}=2.743 min; m/z=384 [M+H]⁺; 406 [M+Na]⁺.

### Example 46

### Preparation of 1-{[2-(1H-indol-3-yl)-ethylcarbamoyl]-methyl}-5-oxo-3,4-diphenyl-2,5-dihydro-1H-pyrrole-2-carboxylic acid allylamide

| | | |
|---|---|---|
| Empirical formula: Molecular weight: | C₃₂H₃₀N₄O₃518.62 g/mol | 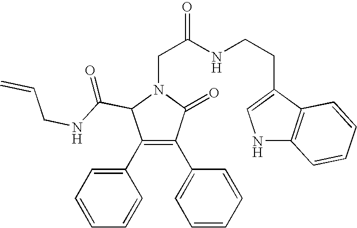 |

HPLC-MS (ESI-TOF): t_{R}=4.058 min; m/z=568 [M+H]⁺.

### Example 47

### Preparation of 3-(4-chloro-phenyl)-1-[2-(3,4-dimethoxy-phenyl)-ethyl]-4-(4-fluoro-phenyl)-5-oxo-2,5-dihydro-1H-pyrrole-2-carboxylic acid tert-butylamide

| | | |
|---|---|---|
| Empirical formula: Molecular weight: | C₃₁H₃₂ClFN₂O₄ 551.06 g/mol | 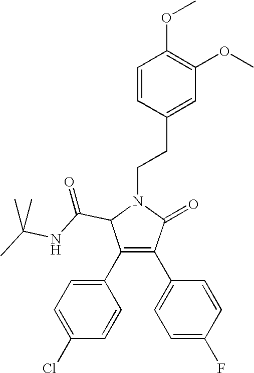 |

HPLC-MS (ESI-TOF): t_{R}=3.946 min; m/z=427 [M+H]⁺; 449 [M+Na]⁺.

### Example 48

### Preparation of 5-(4-but-3-enyl-2-cyclohexylcarbamoyl-1-methyl-5-oxo-2,5-dihydro-1H-pyrrol-3-yl)-2-hydroxy-benzoic acid methyl ester

| | | |
|---|---|---|
| Empirical formula: Molecular weight: | C₂₄H₃₀N₂O₅426.52 g/mol | 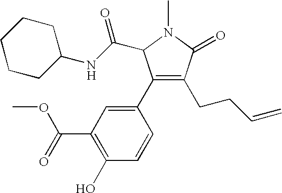 |

HPLC-MS (ESI-TOF): t_{R}=3.556 min; m/z=524 [M+H]⁺;

### Example 49

### Preparation of 3-(4-cyano-phenyl)-1-(3-methyl-butyl)-5-oxo-2,5-dihydro-1H-pyrrole-2-carboxylic acid {[methyl-(2-pyrid-2-yl-ethyl)-carbamoyl]-methyl}-amide

| | | |
|---|---|---|
| Empirical formula: Molecular weight: | C₂₇H₃₁N₅O₃473.58 g/mol | 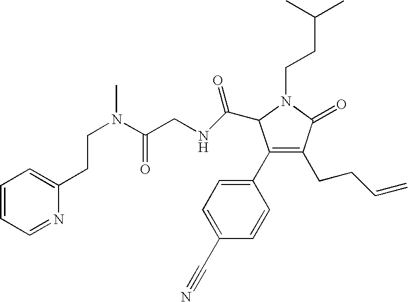 |

HPLC-MS (ESI-TOF): t_{R}=3.334 min; m/z=598 [M+H]⁺; 626 [M+Na]⁺.

### Example 50

### Preparation of 1-(3,3-diphenyl-propyl)-3-(4-methoxy-phenyl)-5-oxo-2,5-dihydro-1H-pyrrole-2-carboxylic acid (4-methoxy-3-oxo-butyl)-amide

| | | |
|---|---|---|
| Empirical formula: Molecular weight: | C₃₂H₃₄N₂O₅526.64 g/mol | 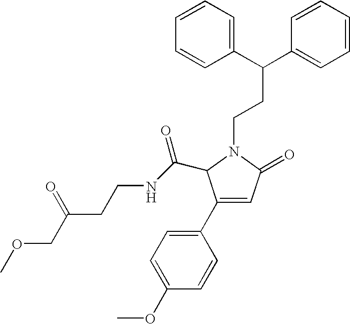 |

HPLC-MS (ESI-TOF): t_{R}=3.115 min; m/z=373 [M+H]⁺; 395 [M+Na]⁺.

### Example 51

### Preparation of 5-oxo-3-phenyl-2,5-dihydro-furan-2-carboxylic acid cyclohexylamide

| | | |
|---|---|---|
| Empirical formula: Molecular weight: | C₁₇H₁₉NO₃285.35 g/mol | 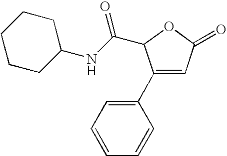 |

HPLC-MS (ESI-TOF): t_{R}=3.472 min; m/z=286 [M+H]⁺; 308 [M+Na]⁺.

### Example 52

### Preparation of 5-oxo-3,4-diphenyl-2,5-dihydro-furan-2-carboxylic acid cyclohexylamide

| | | |
|---|---|---|
| Empirical formula: Molecular weight: Yield: | C₂₃H₂₃NO₃361.44 g/mol 150 mg (41% of theory) | 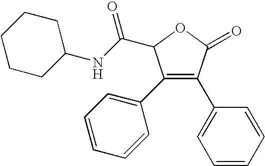 |

HPLC-MS (ESI-TOF): t_{R}=3.732 min; m/z=362 [M+H]⁺; 384 [M+Na]⁺.

¹H NMR (CDCl₃, 400 MHz): δ=7.36 (m, 10H, 10×CH aromatic), 6.30 (d, 1H, NH), 5.81 (s, 1H, (C═O)CH(C—O)), 3.64 (m, 1H, (CH2)2CH(NH)), 1.89 (d, 2H, CH2), 1.67 (m, 2H, CH2), 1.58 (d, 2H, CH2), 1.26 (m, 2H, CH2), 1.12 (m, 2H, CH2).

¹³C NMR (CDCl₃, 100 MHz): δ=171.08 (CO), 163.69 (CO), 157.97 (C, aromatic), 130.38 (C, aromatic), 130.14 (CH, aromatic), 129.25 (CH, aromatic), 129.02 (CH, aromatic), 128.911 (CH, aromatic), 128.52 (CH, aromatic), 128.44 (CH, aromatic), 128.34 (CH, aromatic), 125.62 (CH, aromatic), 80.58 ((CO) CH—O), 48.65 (CH, cyclohexyl), 32.73 (CH2, cyclohexyl), 32.59 (CH2, cyclohexyl), 25.24 (CH2, cyclohexyl), 24.77 (CH2, cyclohexyl).

### Example 53

### Preparation of 2-{[4-(4-fluoro-phenyl)-5-oxo-3-thiophen-2-yl-2,5-dihydro-furan-2-carbonyl]-amino}-propionic acid tert-butyl ester

| | | |
|---|---|---|
| Empirical formula: Molecular weight: Yield: | C₂₂H₂₂FNO₅S 431.49 g/mol 306 mg (47% of theory) | 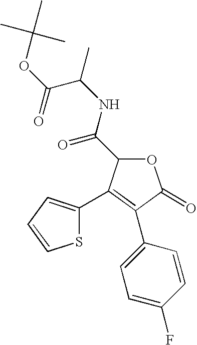 |

HPLC-MS (ESI-TOF): t_{R}=3.781 min; m/z=454 [M+H]⁺; 376 [M-tBu+H]⁺.

¹H-NMR (CDCl₃, 400 MHz): δ=7.76 (d, 1H, (CH)CH(S)), 7.36 (m, 2H, 2×CH aromatic), 7.10 (t, 2H, 2×CH aromatic), 7.00 (m, 1H, CH aromatic), 6.89 (1H, CH aromatic), 5.71 (s, 1H, (CO)CH(O)), 4.34 (q, 1H, (CO)(CH3)CH(NH)), 1.41 (broad, 3H, (CH₃)CH), 1.33 (s, 9H, 3×CH3 tBu).

¹³C NMR (CDCl₃, 100 MHz): δ=171.79 (CO), 165.90 (CO), 165.66 (CO), 151.43 (C, aromatic), 134.76 (2CH, aromatic), 132.80 (2CH, aromatic), 128.38 (1CH, aromatic), 126.18 (aromatic), 117.10 (2CH, aromatic), 83.01 ((CO) CH—O), 49.91 (CH), 28.58 (3CH3), 19.02 (1CH3).

### Example 54

### Preparation of 2-{[4-(3-methoxy-phenyl)-5-oxo-3-phenyl-2,5-dihydro-furan-2-carbonyl]-amino}-4-methyl-pentanoic acid methyl ester

| | | |
|---|---|---|
| Empirical formula: Molecular weight: | C₂₅H₂₇NO₆ 437.5 g/mol | 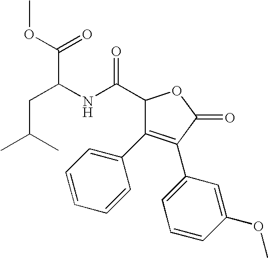 |

HPLC-MS (ESI-TOF): t_{R}=3.746 min; m/z=438 [M+H]⁺; 460 [M+Na]⁺.

¹H NMR (CDCl₃, 400 MHz): δ=7.35 (m, 6H, 6×CH aromatic), 7.25 (m 1H, CH aromatic), 6.92 (m, 2-3, 2×CH aromatic), 6.68 (m, 1H, NH), 5.86 (s, 1H, (C═O)CH(C—O)), 4.55 (m, 1H, (CO)CH(CH2)(NH)), 3.69 (td, 6H, CH3—O-Phe, CH3—O—CO), 1.54 (m, 3H, CH2, CH(CH3)2), 0.88 (m, 6H, 2CH3—O), 0.77 (2d, 6H, 2CH3).

¹³C NMR (CDCl₃, 100 MHz): δ=172.3 (CO), 164,8 (CO), 159.5 (CO), 157.4 (C aromatic, C(OMe)), 130.5 (CH aromatic), 130.4 (CH aromatic), 130.1 (C aromatic), 129.9 (C aromatic), 129.7 (CH aromatic), 129.6 (CH aromatic), 129.0 (CH aromatic), 128.4 (CH aromatic), 121.6 (CH aromatic), 115.1 (CH aromatic), 114.41 (CH, aromatic), 80.35 ((CO)CH(O)), 55.1 (CH3—O—CO), 52.361 (CH3—O), 50.8 ((CO)CH(CH2)(NH)), 41.1 (CH(CH3)2), 24.8 (CH2), 22.8 (CH3), 21.7 (CH3).

### Example 55

### Preparation of 6-oxo-4-phenyl-6H-pyran-2-carboxylic acid tert-butylamide

| | | |
|---|---|---|
| Empirical formula: Molecular weight: | C₁₆H₁₇NO₃271.32 g/mol | 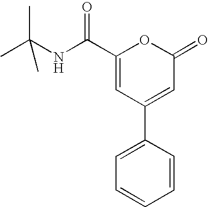 |

HPLC-MS (ESI-TOF): t_{R}=3.418 min; m/z=232 [M−CO₂+H]⁺; 254 [M−CO₂+Na]⁺.

### Example 56

### Preparation of 1-benzyl-6-oxo-4-phenyl-1,2,3,6-tetrahydro-pyridine-2-carboxylic acid tert-butylamide

| | | |
|---|---|---|
| Empirical formula: Molecular weight: Yield: | C₂₃H₂₆N₂O₂362.48 g/mol 341 mg (94% of theory) | 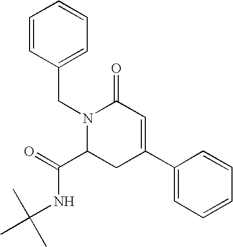 |

HPLC-MS (ESI-TOF): t_{R}=3.584 min; m/z=363 [M+H]⁺; 385 [M+Na]⁺.

¹H NMR (CDC13, 400 MHz): δ=7.39 (m, 10H, 10×CH aromatic), 6.31 (s, 1H, (CO)CH(C)), 5.61 (s, 1H, (CO)(CH2)CH(N)), 4.97 (d, 1H, (Phe)CH2(N)), 4.49 (d, 1H, (Phe)CH2(N)), 4.05 (d, 1H, (CH)CH2(C)), 3.40 (d(CH)CH2(C)), 2.90 (m, 1H), 1.17 (s, 3H), 1.12 (s, 9H, 3×CH3 tBu)).

¹³C NMR (CDC13, 100 MHz): δ=169.3 (CO), 164.5 (CO), 149.6 (C aromatic), 137.1 (C aromatic), 129.7 (CH aromatic), 129.0 (CH aromatic), 128.7 (CH aromatic), 128.6 (CH aromatic), 128.1 (CH aromatic), 126.2 (CH aromatic), 118.8 (CH double bond), 59.7 ((CO)CH(N)(CH2)), 51.4 (C(Me)3), 50.0 ((Phe)CH2(N)), 30.3 ((CH)(CH2)(C)), 28.3 (3×CH3 tBu).

### Example 57

### Preparation of 2-hydroxy-4-(3-methoxy-phenyl)-5-oxo-3-phenyl-2,5-dihydro-furan-2-carboxylic acid cyclohexyl-amide

| | | |
|---|---|---|
| Empirical formula: Molecular weight: | C₂₄H₂₅NO₅ 407.47 g/mol | 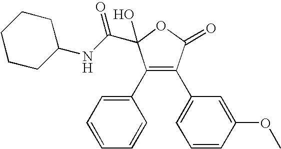 |

HPLC-MS (ESI-TOF): t_{R}=3.736 min; m/z=408 [M+H]⁺; 430 [M+Na]⁺.

### Example 58

### Preparation of 1-benzyl-5-oxo-3,4-diphenyl-2,5-dihydro-1H-pyrrole-2-carboxylic acid tert-butylamide

| | | |
|---|---|---|
| Empirical formula: Molecular weight: | C₂₈H₂₈N₂O₂424.55 g/mol | 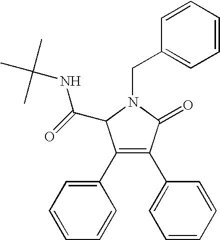 |

HPLC-MS (ESI-TOF): t_{R}=3.950 min; m/z=441 [M+H]⁺; 463 [M+Na]⁺.

¹H NMR (CDC13, 400 MHz): δ=7.49 (2H, aromatic), 7.41 (2H, aromatic), 7.29 (12H, aromatic), 5.47 (d, 2H, (Phe)CH2(N)), 4.81 (d, 1H), 4.36 (d, 1H), 1.58 (s, 1H), 1.24 (s, 1H), 0.91 (s, 9H, tBu).

1. Compounds of the general formula (I): 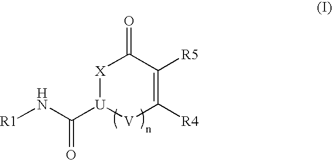
wherein
X is an oxygen atom or a group of formula NR6;
n is 0 or 1;
U is CH or COH;
V is a group of formula CR2R3, or U-V together are a group of formula 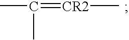
R1 is an alkyl, heteroalkyl, aryl, heteroaryl, cycloalkyl, heterocycloalkyl, aralkyl or heteroaralkyl radical;
R2 is a hydrogen atom, an alkyl, heteroalkyl, aryl, heteroaryl, cycloalkyl, heterocycloalkyl, aralkyl or heteroaralkyl radical;
R3 is a hydrogen atom, an alkyl, heteroalkyl, aryl, heteroaryl, cycloalkyl, heterocycloalkyl, aralkyl or heteroaralkyl radical;
R4 is a hydrogen atom, an alkyl, heteroalkyl, aryl, heteroaryl, cycloalkyl, heterocycloalkyl, aralkyl or heteroaralkyl radical;
R5 is a hydrogen atom, a halogen atom, a hydroxy group, an alkyl, heteroalkyl, aryl, heteroaryl, cycloalkyl, heterocycloalkyl, aralkyl or heteroaralkyl radical;
R6 is a hydrogen atom, an alkyl, heteroalkyl, aryl, heteroaryl, cycloalkyl, heterocycloalkyl, aralkyl or heteroaralkyl radical;
or R2 and R3, or R2 and R4, together are part of a cycloalkyl, heterocycloalkyl, aralkyl or heteroaralkyl ring system;
or a pharmacologically acceptable salt, solvate, hydrate or a pharmacologically acceptable formulation thereof.

2. Compounds according to claim 1, wherein X is an oxygen atom.

3. Compounds according to claim 1, wherein X is a group of formula NR6.

4. Compounds according to one of claims 1 to 3, wherein U is CH.

5. Compounds according to one of claims 1 to 3, wherein U is COH.

6. Compounds according to one of claims 1 to 5, wherein n is 0.

7. Compounds according to one of claims 1 to 5, wherein n is 1.

8. Compounds according to one of claims 1 to 3 and 7, wherein U-V together are a group of formula 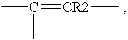
wherein R2 is as defined in one of the previous claims.

9. Pharmaceutical compositions that comprise a compound according to one of claims 1 to 8 and, optionally, carrier substances and/or adjuvants.

10. Use of a compound or of a pharmaceutical composition according to one of claims 1 to 9 for the inhibition of kinases.

11. Use of a compound or of a pharmaceutical composition according to one of claims 1 to 9 for the treatment and/or prevention of diseases mediated by kinase activity.

12. Use of a compound or of a pharmaceutical composition according to one of claims 1 to 9 for the treatment and/or prevention of inflammatory diseases and of diseases caused by autoimmune reactions.

13. Use of a compound or of a pharmaceutical composition according to one of claims 1 to 9 for the treatment and/or prevention of rheumatoid arthritis, asthma, MDR (multiple drug resistance), COPD, ARDS, cancer, stroke, Alzheimer's, osteoarthritis, lung disease, septic shock, angiogenesis, dermatitis, and also for in vivo stimulation of nerve growth, in vivo inhibition of scar tissue formation and/or in vivo reduction of secondary damage.

14. Method for the preparation of compounds according to one of claims 1-8, characterised in that compounds of formulae (II), (III), (IV) and, where appropriate, (V) are reacted with one another: 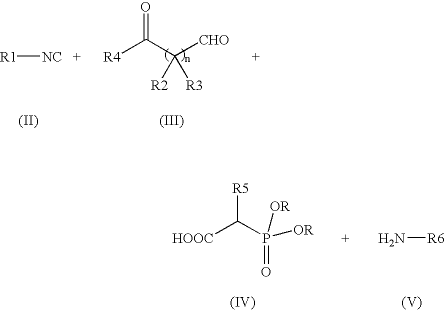
the radicals being as defined in one of the previous claims.

15. Method according to claim 14, characterised in that the reaction is carried out in an aprotic solvent.

16. Method according to one of claims 14 or 15, characterised in that an ether is used as solvent.

17. Method according to one of claims 14 to 16, characterised in that the reaction is carried out at a temperature of from −80 to 120° C., preferably from 0 to 25° C.

18. Method according to one of claims 14 to 17, characterised in that, in a first step, the compounds of formulae II, III and IV and, where appropriate, V are reacted with one another and, in a second step, the reaction product from the first step is reacted in the presence of at least one alkali metal salt, such as a lithium salt.

19. Method according to one of claims 14 to 18, characterised in that the second step is carried out in the presence of at least one base, such as a tertiary basic amine.

20. Method according to one of claims 14 to 19, characterised in that the reaction is carried out in the presence of or with exclusion of O₂.

21. Use of a method according to one of claims 14 to 20 for the synthesis of compound libraries.

22. Use of a method according to one of claims 14 to 20 for the finding of lead structures.
